(19) United States
(12) Patent Application Publication
(10) Pub. No.: US 2009/0263829 A1
(43) Pub. Date: Oct. 22, 2009

(54) ALZHEIMER'S DISEASE BIOMARKERS AND METHODS OF USE

(75) Inventors: David Holtzman, St. Louis, MO (US); Yan Hu, Foster City, CA (US); Robert Reid Townsend, St. Louis, MO (US)

Correspondence Address: POLSINELLI SHUGHART PC, 100 SOUTH FOURTH STREET, SUITE 100, SAINT LOUIS, MO, 63102-1825, US

(73) Assignee: WASHINGTON UNIVERSITY IN ST. LOUIS, St. Louis, MO (US)

(21) Appl. No.: 12/282,799

(22) PCT Filed: Mar. 12, 2007
(86) PCT No.: PCT/US07/63762

§ 371 Date: Feb. 18, 2009

Related U.S. Application Data
(60) Provisional application No. 60/782,175, filed on Mar. 14, 2006.

(51) Int. Cl. G01N 33/573; C07K 14/00; C12N 9/48; G01N 33/53

(52) U.S. Cl. 435/7.4; 530/350; 435/212; 435/7.1

(57) ABSTRACT

The invention encompasses biomarkers for AD, a method for detecting AD, a method of monitoring AD, and a kit for quantifying biomarkers for AD.

### GOVERNMENTAL RIGHTS

This invention was made with government support under AG025662 awarded by the NIH, The government has certain rights in the invention.

### HELP OF THE INVENTION

The invention generally relates to biomarkers for Alzheimer's disease, methods of detecting Alzheimer's disease, methods of monitoring Alzheimer's disease, and kits for detecting biomarkers for Alzheimer's disease,

### BACKGROUND OF THE INVENTION

Alzheimer's disease (AD) will likely become the greatest public health crisis in the United States within the next 2-3 decades if left unchecked. There are currently no proven treatments that delay the onset or prevent the progression of AD, although a few promising candidates are being developed. During the development of these therapies, it will be very important to have biomarkers that can identify individuals at high risk for AD or at the earliest clinical stage of AD in order to target them for therapeutic trials, disease-modifying therapies and to monitor their therapy. Clinicopathological studies suggest that AD pathology (particularly the buildup of amyloid plaques) begins 10-20 years before cognitive symptoms. Even the earliest clinical symptoms of AD are accompanied by, and likely due to neuronal/synaptic dysfunction and/or cell death. Thus, it will be critical to identify individuals with “preclinical” and very early stage AD, prior to marked clinical symptoms and neuronal loss, so new therapies will have the greatest clinical impact.

A definitive diagnosis of AD can still only be obtained via neuropathologic evaluation at autopsy. Investigators at the Washington University School of Medicine (WUSM) developed a Clinical Dementia Rating (CDR) scale in which an individual's cognition is rated as normal (CDR 0), or demented with severities of very mild, mild, moderate or severe (CDR 0.5, 1, 2 or 3, respectively) (See Morris, Neurology, 1993; 43:2412, hereby included by reference). individuals diagnosed with possible/probable dementia of the Alzheimer's type (DAT) are usually CDR 1 or greater. One challenge has been to diagnose individuals at earlier stages, when clinical symptoms are less severe. During these early stages (CDR 0.5, often lasting 2-5 years or longer), the majority of individuals meet clinical criteria for mild cognitive impairment (MCI) (Peterson et al. Arch. Neurol, 1899; 58:303). Data suggest an early and insidious pathogenesis of AD. the clinical manifestation of which becomes apparent only after substantial neuronal cell death and synapse loss has taken place. These findings have profound implications for AD therapeutic and diagnostic strategies.

At present, a few AD biomarkers have been identified that may differentiate individuals with clinical disease (i.e., DAT) from those who are cognitively normal. Mean cerebral spinal fluid (CSF) amyloid beta (Aβ42) levels have been consistently reported to be decreased in AD, including cases of mild dementia, although this decrease may not be specific for AD. CSF Aβ42 is also decreased in MCI, but there is great overlap with control group values. Many studies have reported elevated levels of CSF total tau (and phosphorylated forms) in AD patients. However, similar to Aβ42, there is significant overlap between individual tau values in MCI/AD and control groups, and this increase is not specific for AD. In addition to Aβ42 and tau, differences in other candidate AD biomarkers that likely reflect CNS damage have been observed, including isoprostanes, and 4-hydroxy-2-nonenal (markers of oxidative damage), and sulfatide, a sphingolipid produced by oligodendrocytes. To date, however, none of these individual candidate markers have achieved levels of sensitivity and specificity acceptable for use in disease diagnosis.

### SUMMARY OF THE INVENTION

One aspect of the invention encompasses a biomarker for AD. The biomarker comprises the level of antithrombin III in a bodily fluid of a subject.

Another aspect of the invention encompasses a method for detecting AD. Generally speaking, the method comprises quantifying the level of antithrombin III in a bodily fluid of the subject and determining if the quantified level of antithrombin III is elevated in comparison to the average antithrombin III level for a subject with a CDR of 0.

Yet another aspect of the invention encompasses a method for monitoring AD. Typically, the method comprises quantifying the level of antithrombin III in a bodily fluid of the subject and comparing the quantified level of antithrombin III to a previously quantified antithrombin III level of the subject.

Still another aspect of the invention encompasses a kit for quantifying antithrombin III in a bodily fluid of a subject. The kit comprises the means to quantify antithrombin III and instructions.

### BRIEF DESCRIPTION OF THE FIGURES

FIG. 1 depicts the 2-D DIGE analysis of CSF prior to and following depletion of high abundance proteins. The same amount of protein (19 micrograms) in CSF prior to depletion (A) and following depletion (B) and in the retained proteins (C) was labeled with Cy2 (blue), Cy3 (green), and Cy5 (red), respectively, and analyzed on a single gel (10% isocratic SDS-PAGE gel). (D) overlay of all three fluorescent images demonstrates the position of the depleted proteins (pink) with respect to the low abundance proteins revealed by the depletion method.

FIG. 2 depicts a representative 2-D DIGE image (Cy2-labeled) of CSF that has been depleted of six high abundance proteins. 50 micrograms of protein was labeled and resolved first on a pH 3-10 IPG strip and further separated on a 10-20% gradient SDS-PAGE gel.

FIG. 3 depicts representative gel images and 3-D representations of one of the apoE spots that displayed intraindividual variation. Shown here are the data from Subject 2. There is a 3.1-fold change of the levels of this apoE spot between the two time points. (A) represents timepoint 1; (B) represents timepoint 2.

FIG. 4 depicts the hierarchical clustering of the 2-D DIGE profiles of 306 matched proteins spots from the 12 CSF samples from six individual subjects at time 1 (T1) and 2 weeks (T2). Each 2-D DIGE profile (column) contains 306 matched protein spots. Lines correspond to individual proteins, and colors represent their standard abundance after a log transformation and Z-score normalization (red, more abundant; green, less abundant). The CDR 0.5 samples are marked with an asterisk. Spotfire software was used to generate the cluster tree and the heat map. Distance in the cluster tree depicted here is not a reflection of similarity or strength of association.

FIG. 5 depicts the multidimensional scaling analysis of the 2-D DIGE profiles of 12 CSF samples from six individual subjects at time 1 (T1) and then 2 weeks later (T2). A 2-D projection of the 3-D scatter plot is shown. The proteomic profile of each sample is represented by a point. The axes correspond to the first three principal components. A single color has been used to label two intraindividual CSF samples.

FIG. 6 depicts graphs showing the CSF levels of biomarkers in CDR group of 0, 0.5, and 1. The graphs were generated using unadjusted raw data. One-way ANOVA analysis was performed to compare the average levels of candidate biomarkers in the three groups and where overall p<0.05, Bonferroni's multiple comparison test was done to examine which comparisons generate statistically significant differences (denoted by asterisks). (A) ACT; (B) ATIII; (C) ZAG; (D) CDNP1.

FIG. 7 depicts graphs showing that the mean levels of CSF Aβ₄₂ are decreased (A) and levels of total tau are increased (B) in very mild AD vs. control subjects. Clinical dementia rating (CDR) 0 equals no cognitive impairment, CDR 0.5 represents very mild dementia, and CDR 1 represents mild dementia due to AD. P values calculated using the raw data (P) and those calculated using the log-transformed and adjusted dataset (P*) are also displayed,

FIG. 8 depicts graphs showing the levels of selected candidate biomarkers in a large CSF sample set as assayed by ELISA. P values calculated using the raw data (P) and those calculated using the log-transformed and adjusted dataset (P*) are displayed. ACT, ATIII, and ZAG are significantly increased in AD (CDR 0.5 and 1) vs. control (CDR 0) samples. (A) ACT; (8) ZAG; (C) Gelsolin; (D) ATIII; (E) CDNP1; (F) AGT.

FIG. 9 depicts graphs showing that the levels of ACT, ATIII, and ZAG are not significantly different in plasma between AD (CDR 0.5 and 1) vs. control (CDR 0) samples. (A) ACT; (B) ATIII; (C) ZAG

FIG. 10 depicts graphs showing the correlations between the CSF and plasma levels of candidate biomarkers, including ACT (A), ATIII (B), and ZAG (C).

FIG. 11 depicts a graph showing the receiver operating characteristic curve (ROC) for the normalized and adjusted CSF concentrations of each biomarker candidate and the optimum linear combination (Optimum) combining data from all biomarkers.

### DETAILED DESCRIPTION OF THE PREFERRED EMBODIMENTS

Given the early, insidious pathogenesis of AD, combined with the theory that neuronal degeneration is easier to slow or halt than to reverse, it is vital to identify biomarkers that can detect the disease before or during the early development of symptoms and irreversible pathologic damage. Such biomarkers could be used for AD screening and diagnosis, as well as potentially for assessing response to new therapies. Despite the existence of a few promising CSF biomarkers for early stage AD as described above, these candidate markers have not fulfilled the consensus criteria necessary for use in individual diagnosis. Given the probability of multiple underlying pathogenic mechanisms of late-onset AD, it is likely that a battery of biomarkers will be more useful than an individual marker. Therefore, new and better biomarkers must be identified.

To this end, the present invention provides novel AD biomarkers present in the bodily fluid of a subject. The level of these biomarkers correlate with CDR score, and therefore may allow a more accurate diagnosis or prognosis of AD in subjects that are at risk for AD, that show no clinical signs of AD, or that show minor clinical signs of AD. Furthermore, the biomarkers may allow the monitoring of AD, such that a comparison of biomarker levels allows an evaluation of disease progression in subjects that have been diagnosed with AD, or that do not yet show any clinical signs of AD. Moreover, the AD biomarkers of the invention may be used in concert with known AD biomarkers such that a more accurate diagnosis or prognosis of AD may be made.

### I. Biomarkers to Detect Alzheimer's Disease

One aspect of the present invention provides biomarkers to detect AD. A biomarker is typically a protein, found in a bodily fluid, whose level varies with disease state and may be readily quantified. The quantified level may then be compared to a known value. The comparison may be used for several different purposes, including but not limited to, diagnosis of AD, prognosis of AD, and monitoring treatment of AD.

Through proteomic screening performed as detailed in the examples, several novel biomarkers have been identified for AD. In one embodiment, the level of a serine protease inhibitor is a biomarker for AD. Examples of serine protease inhibitors include alpha 1-antitrypsin, alpha 1-antichymotrypsin, alpha 2-antiplasmin, antithrombin III, complement 1-inhibitor, neuroserpin, plasminogen activator inhibitor-1 and 2, and protein Z-related protease inhibitor (ZPI). In another embodiment the biomarker is the level of α1-antichymotrypsin (ACT). In yet another embodiment, the biomarker is the level of antithrombin III (ATIII). In an alternative embodiment, the biomarker is the level of zinc-alpha-2-glycoprotein (ZAG). In another alternative embodiment, the biomarker is the level of carnosinase 1 (CNDP1).

Each of the biomarkers identified above may be used in concert with another biomarker for purposes including but not limited to diagnosis of AD, prognosis of AD, and monitoring treatment of AD. For instance, two or more, three or more, four or more, five or more, or six or more AD biomarkers may be used in concert. As explained above, there are several known biomarkers for AD. In one embodiment, two or more biomarkers from the group comprising ACT, ATIII, ZAG, CNDP1, Aβ42 and tau are used in concert. In yet another embodiment, three or more biomarkers from the group comprising ACT, ATIII, ZAG, CNDP1, Aβ42 and tau are used in concert. In still another embodiment, four or more biomarkers from the group comprising ACT, ATIII, ZAG, CNDP1, Aβ42and tau are used in concert. In yet still another embodiment, ACT, ATIII, ZAG, CNDP1, Aβ42 and tau are used in concert as biomarkers for AD.

a. Bodily Fluids

The levels of AD biomarkers of the invention may be quantified in several different bodily fluids. Non-limiting examples of bodily fluid include whole blood, plasma, serum, bile, lymph, pleural fluid, semen, saliva, sweat, urine, and CSF. In one embodiment, the bodily fluid is selected from the group comprising whole blood, plasma, and serum. In another embodiment, the bodily fluid is whole blood. In yet another embodiment, the bodily fluid is plasma. In still yet another embodiment, the bodily fluid is serum. In an exemplary embodiment, the bodily fluid is CSF.

As will be appreciated by a skilled artisan, the method of collecting a bodily fluid from a subject can and will vary depending upon the nature of the bodily fluid. Any of a variety of methods generally known in the art may be utilized to collect a bodily fluid from a subject. Generally speaking, the method preferably maintains the integrity of the AD biomarker such that it can be accurately quantified in the bodily fluid. One method of collecting CSF is detailed in the examples. Methods for collecting blood or fractions thereof are well known in the art. For example, see U.S. Pat. No. 5,286,262, which is hereby incorporated by reference in its entirety.

A bodily fluid may be tested from any mammal known to suffer from Alzheimer's disease or used as a disease model for Alzheimer's disease. In one embodiment, the subject is a rodent Examples of rodents include mice, rats, and guinea pigs. In another embodiment, the subject is a primate. Examples of primates include monkeys, apes, and humans. In an exemplary embodiment, the subject is a human. In some embodiments, the subject has no clinical signs of AD. In other embodiments, the subject has mild clinical signs of AD, for instance, corresponding to a CDR score of 0.5. In yet other embodiments, the subject may be at risk for AD. In still other embodiments, the subject has been diagnosed with AD.

b. Level of Biomarker

The level of the biomarker may encompass the level of protein concentration or the level of enzymatic activity. In either embodiment, the level is quantified, such that a value, an average value, or a range of values is determined In one embodiment, the level of protein concentration of the AD biomarker is quantified. In another embodiment the concentration of ATIII is quantified. In yet another embodiment, the concentration of ACT is quantified. In still another embodiment, the concentration of ZAG is quantified. In still yet another embodiment, the concentration of CNDP1 is quantified.

There are numerous known methods and kits for measuring the amount or concentration of a protein in a sample, including ELISA, western blot, absorption measurement, colorimetric determination, Lowry assay, Bicinchoninic acid assay, or a Bradford assay. Commercial kits include ProteoQwest™ Colorimetric Western Blotting Kits (Sigma-Aldrich, Co.), QuantiPro™ bicinchoninic acid (BCA) Protein Assay Kit (Sigma-Aldrich, Co.), FluoroProfile™ Protein Quantification Kit (Sigma-Aldrich, Co.), the Coomassie Plus—The Better Bradford Assay (Pierce Biotechnology, Inc.), and the Modified Lowry Protein Assay Kit (Pierce Biotechnology, Inc.). In certain embodiments, the protein concentration is measured by ELISA. For instance, the level of ATIII may be quantified by ELISA as described in the examples.

In another embodiment, the level of enzymatic activity of the biomarker is quantified. Generally, enzyme activity may be measured by means known in the art, such as measurement of product formation, substrate degradation, or substrate concentration, at a selected point(s) or time(s) in the enzymatic reaction. In one embodiment, the enzyme activity of ATIII is quantified. In another embodiment, the enzyme activity of ACT is quantified. In yet another embodiment, the enzyme activity of CNDP1 is quantified. There are numerous known methods and kits for measuring enzyme activity. For example, see U.S. Pat. No. 5,854,152. Some methods may require purification of the AD biomarker prior to measuring the enzymatic activity of the biomarker. A pure biomarker constitutes at least about 90%, preferably, 95% and even more preferably, at least about 99% by weight of the total protein in a given sample. AD biomarkers of the invention may be purified according to methods known in the art, including, but not limited to, ion-exchange chromatography, size-exclusion chromatography, affinity chromatography, differential solubility, differential centrifugation, and HPLC. (See Current Protocols in Molecular Biology, Eds. Ausubel, et al., Greene Publ. Assoc., Wiley-Interscience, New York)

### II. Methods of Using the Biomarkers

a. Using Biomarkers for the Diagnosis or Prognosis of AD

In one embodiment, the invention encompasses a method for detecting AD comprising quantifying the level of an AD biomarker in a bodily fluid of a subject and subsequently determining if the quantified level of the biomarker is elevated or depressed in comparison to the average level of the biomarker for a subject with a CDR of 0. The subject may have no clinical signs of AD, the subject might be at risk for AD, or alternatively, the subject might show mild dementia (CDR of 0.5). The average level of the biomarker for a subject with a CDR of 0 refers to the arithmetic average of the biomarker level in a bodily fluid of at least 50 subjects with a CDR of 0.

An elevated or depressed biomarker level may lead to either a diagnosis or prognosis of AD. In one embodiment, an elevated biomarker level indicates a diagnosis of AD. In another embodiment, an elevated biomarker level indicates a prognosis of AD. In yet another embodiment, a depressed biomarker level indicates a diagnosis of AD. In still yet another embodiment, a depressed biomarker level indicates a prognosis of AD.

A skilled artisan will realize that whether an elevated or a depressed biomarker level in comparison to the average level of the biomarker for a subject with a CDR of 0 is indicative of AD will depend on the biomarker in question. In one embodiment, an elevated level of ATIII indicates a diagnosis or prognosis of AD. In another embodiment, an elevated level of ACT indicates a diagnosis or prognosis of AD. In yet another embodiment, an elevated level of tau indicates a diagnosis or prognosis of AD. In still another embodiment, a depressed level of Aβ42 indicates a diagnosis or prognosis of AD. In yet still another embodiment, a modulated level of ZAG indicates a diagnosis or prognosis of AD. In an alternative embodiment, a depressed level of CNDP1 indicates a diagnosis or prognosis of AD.

An AD biomarker of the invention may be quantified in concert with another known AD biomarker as detailed in Part I above. For example, ATIII may be used as an AD biomarker in concert with Aβ42. In this example, a simultaneously elevated ATIII level and a depressed Aβ42 level in a bodily fluid of a subject would be indicative of a diagnosis or prognosis of AD.

The percent elevation or depression of an AD biomarker compared to the average level of the biomarker for a subject with a CDR of 0 is typically greater than 15% to indicate a diagnosis or prognosis of AD. In some instances, the percent elevation or depression is 15%, 16%, 17%, 18%, 19%, 20%, 21%, or 22%. In other instances, the percent elevation or depression is 23%, 24%, 25%, 26%, 27%, 28%, 29% or 30%. In still other instances, the percent elevation or depression is greater than 30%. In alternative instances, the percent elevation or depression is greater than 50%.

b. Using Biomarkers to Monitor AD

Another embodiment of the invention encompasses a method for monitoring AD comprising quantifying the level of an AD biomarker in a bodily fluid of a subject and comparing the quantified level of the biomarker to a previously quantified biomarker level of the subject to determine if the quantified level is elevated or depressed in comparison to the previous level. The subject may be diagnosed with AD, or alternatively, may have no clinical signs of AD. The comparison may give an indication of disease progression. Therefore, the comparison may serve to measure the effectiveness of a chosen therapy. Alternatively, the comparison may serve to measure the rate of disease progression. For example, a depressed ATIII level, in comparison to a previous level, may indicate an abatement of disease progression.

In the context of monitoring AD, the percent elevation or depression of an AD biomarker compared to a previous level may be from 0% to greater than about 50%. In one embodiment, the percent elevation or depression is from about 1% to about 10%. In another embodiment, the percent elevation or depression is from about 10% to about 20%. In yet another embodiment, the percent elevation or depression is from about 20% to about 30%. In still another embodiment, the percent elevation or depression is from about 30% to about 40%. In yet still another embodiment, the percent elevation or depression is from about 40% to about 50%. In a further embodiment, the percent elevation or depression is greater than 50%.

### III. Kits for Detecting or Monitoring AD

Another aspect of the invention encompasses kits for detecting or monitoring AD in a subject. A variety of kits having different components are contemplated by the current invention. Generally speaking, the kit will include the means for quantifying one or more AD biomarkers in a subject. In another embodiment, the kit will include means for collecting a bodily fluid, means for quantifying one or more AD biomarkers in the bodily fluid, and instructions for use of the kit contents. In certain embodiments, the kit comprises a means for quantifying AD biomarker enzyme activity. Preferably, the means for quantifying biomarker enzyme activity comprises reagents necessary to detect the biomarker enzyme activity. In certain aspects, the kit comprises a means for quantifying the amount of AD biomarker protein. Preferably, the means for quantifying the amount of biomarker protein comprises reagents necessary to detect the amount of biomarker protein.

In one embodiment, the kit comprises means to quantify the level of ATIII in a bodily fluid of a subject. The level of ATIII refers to either the enzyme activity of ATIII or the protein concentration of ATIII. In another embodiment, the kit comprises a means to quantify the level of at least one AD biomarker. In yet another embodiment, the kit comprises means to quantify the level of at least two, at least three, at least four, at least five or at least six AD biomarkers. In still yet another embodiment, the kit comprises means to quantify the level of at least seven, at least eight, at least nine, or at least ten AD biomarkers. In still another embodiment, the kit comprises means to quantify the level of ten or more biomarkers. In certain embodiments, the kit comprises the means to quantify the level of one or more biomarkers from the group consisting of ATIII, ACT, ZAG, CNDP1, Aβ42, and tau. In each of the above embodiments, the AD biomarker level refers to either the biomarker enzyme activity or the biomarker protein concentration. The means necessary to detect either enzyme activity or protein concentration are discussed in Part II above.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples illustrate the invention.

### Example 1

### Materials and Methods for Example 1

CSF Sampling: Human CSF samples were obtained by lumbar puncture (LP) from subjects enrolled in the Memory and Aging Project at Washington University as part of an ongoing biomarker study. The study protocol was approved by the Human Studies Committee at Washington University, and written and verbal informed consent was obtained from each participant at enrollment. In 6 individuals, two samples were obtained from the same individual 2 weeks apart. Two weeks is an arbitrary time span, chosen to allow the skin, subcutaneous tissue, and meninges to have adequate time for repair prior to a second LP. Other time periods may certainly be used. All LPs were performed at the same time of the day with no fasting requirement. 25-35 ml of CSF was obtained from all participants with either a 22 or a 25 gauge spinal needle. All CSF samples were free of blood contamination. After collection, CSF samples were briefly centrifuged at 1,000×g to pellet any cell debris, frozen, and stored in polypropylene tubes at −80° C. in 0.5-ml aliquots until analysis. The age of these six individuals ranged from 64 to 91 years. The cognitive state of the subjects was rated using a Clinical Dementia Rating (CDR) scale in which an individual's cognition is rated as normal (CDR 0), or demented with severities of very mild, mild, moderate or severe (CDR 0.5, 1, 2 or 3, respectively). Individuals diagnosed with possible/probable dementia of the Alzheimer's type (DAT) are usually CDR 1or greater. Of the 8 individuals who had CSF sampling on 2 occasions, 2 weeks apart, four of these subjects had a CDR score of 0, and the other two were rated as CDR 0.5. The protein content in each CSF sample was determined with the micro-BCA protein assay kit (Pierce), and it ranged from 570 to 1,000 μg/ml.

Multiaffinity Immunodepletion of CSF Proteins: Because albumin, IgG, α1-antitrypsin, IgA, transferrin, and haptoglobin collectively account for ˜80% of the total CSF protein content, these proteins were selectively removed to enrich for proteins of lower abundance. An antibody-based multiaffinity removal system (Agilent Technologies, Palo Alto, Calif.) was used according to the manufacturers instructions. Briefly 1.5-2 ml of CSF was concentrated and buffer-exchanged with Agilent Buffer A to a final volume of 50 μl using Amicon Ultra-4 centrifugal filter units (10-kDa cut-off) (Millipore). Samples were then diluted to 200 μl with Buffer A and passed through an Ultra-free MC microcentrifuge filter (0.22 μm) (Millipore) to remove particulates. The filtrate was injected at 0.25 ml/min onto a 4.8×50-mm multiple affinity removal column equilibrated at room temperature with Agilent Buffer A on a Microtech (Vista, Calif.) Ultra-Plus HPLC system. CSF devoid of high abundance proteins (flow-through) was collected between 1.5 and 6 min. After 9 min of elution with Buffer A, the eluant was changed to Agilent Buffer B at 1 ml/min. The six bound proteins were eluted from the column between 10 and 14 min. After 3.5 min, the column was regenerated with Buffer A.

2-D DIGE: Depleted CSF samples were buffer-exchanged and concentrated with lysis buffer (30 mM Tris-Cl, pH 7.8, 7 M urea, 2 M thiourea, 4% CHAPS containing protease inhibitors (catalog number 697498, Roche Diagnostics) and phosphatase inhibitors (catalog numbers 524624 and 524625, EMD Biosciences, Darmstadt, Germany) using Amicon Ultra-4 centrifugal filter units (10-kDa cut-off) (Millipore). The protein concentration was determined with a modified Lowry method (PlusOne 2D-Quant kit, Amersham Biosciences). Fifty micrograms of protein from each sample was labeled with 400 pmol of one of three N-hydroxysuccinimide cyanine dyes for proteins (Amersham Biosciences), diluted with rehydration buffer (7 M urea, 2 M thiourea, 4% CHAPS, 2.5% DTT, 10% isopropanol, 5% glycerol, and 2% PharmalytepH 3-10), combined according to experimental design, and equilibrated with IPG strips (24 cm; pH 3-10, nonlinear). The three samples that were equilibrated with each IPG strip consisted of two depleted CSF samples from the same individual (Cy2 and Cy5) and a pooled sample (pooled using an equal volume aliquot of each of the 12 CSF samples) (Cy3) as the internal standard. First dimension isoelectric focusing was performed at 65.6 kV-h in an Ettan IPGphor system (Amersham Biosciences). The strips were then treated with reducing and alkylating solutions prior to the second dimension (SDS-PAGE). After equilibration with a solution containing 6 M urea, 30% glycerol, 2% SDS, 50 mM Tris-Cl, pH 7.8. 32 mM DTT, the strips were treated with the same solution containing 325 mM iodoacetamide instead of DTT. The strips were overlayered onto a 10% isocratic or gradient SDS-PAGE gel (20×24 cm), immobilized to a low fluorescence glass plate and electrophoresed for ˜18 h at 1 watt/gel. The Cy2-, Cy3-, and Cy5-labeled images were acquired on a Typhoon 9400 scanner (Amersham Biosciences) at the excitation/emission values of 488/520, 532/580, 633/870 nm, respectively.

Image Analyses: Intragel spot detection and quantification and intergel matching and quantification were performed using Differential In-gel Analysis (DIA) and Biological Variation Analysis (BVA) modules of DeCyder software version 5.01 (Amersham Biosciences) as described previously (Alban et al., (2003) Proteomics 3, 36-44; Tonge et al., (2001) Proteomics 1, 377-396). Briefly in DIA, the Cy2, Cy3, and Cy5 images for each gel were merged, spot boundaries were automatically detected, and normalized spot volumes (protein abundance) were calculated. During spot detection, the estimated number of spots was set at 3,500, and the exclude filter was set as follows; slope, >1.1; area, <100; peak height, <100; and volume, <10.000. This analysis was used to calculate abundance differences in given proteins between two samplings from the same individual. The resulting spot maps were exported to BVA. Matching of the protein spots across six gels was performed after several rounds of extensive land marking and automatic matching. Dividing each Cy2 or Cy5 spot volume with the corresponding Cy3 (internal standard) spot volume within each gel gave a standard abundance, thereby correcting intergel variations. For each of the CSF samples, a profile was created that consisted of standard abundance for all of the matched spots.

Protein Digestion and Mass Spectrometry: Gel features were selected in the DeCyder software and the X and Y coordinates were saved in a file for spot excision. After translation using in-house software (imagemapper), the central core (1.8 mm) of the selected gel features was excised with a ProPic robot (Genomics Solutions, Ann Arbor, Mich.) and transferred to a 96-well PGR plate. The gel pieces were then digested in situ with trypsin using a modification of a published method (Havlis et al., (2003) Anal. Chem. 75, 1300-1306). To maximize specificity, sensitivity, and sequence coverage of the digested proteins, the resulting peptide pools were analyzed by tandem MS using both MALDI and ESI. Spectra of the peptide pools were obtained on a MALDI-TOF/TOF instrument (Proteomics 4700, Applied Biosystems, Foster City, Calif.). The initial spectra were used to determine the molecular weights of the peptides (to within 20 ppm of their theoretical masses). Selected precursor ions were then focused in the instrument using a timed ion selector, and peptide fragmentation spectra were produced after high energy (1.5-keV) collision-induced dissociation. ESI-MS was performed using an advanced capillary LC-MS/MS system (Eksigent nano-LC 1D Proteomics, Eksigent Technologies, Livermore, Calif.). A nanoflow (200 nl/min) pulse-free liquid chromatograph was interfaced to a quadrupole time-of-flight mass spectrometer (Q-STAR XL, Applied Biosystems) using a PicoView system (New Objective, Woburn, Mass.). Sample injection was performed with an Endurance autosampler (Spark Holland, Plainsboro, N.J.). The peptide fragmentation spectra were processed using Data Explorer version 4.5 or Analyst software (Applied Biosystems). After centroiding and background subtraction, the peak lists were used to search databases with MASCOT version 1.9 (Matrix Sciences, Boston, Mass.). Peptide sequences were qualified by manual interpretation of raw non-centroided spectra.

CSF Biomarker Assessment (ELISA): CSF samples were analyzed for total tau, amyloid β42 (Aβ42), α1-chymotrypsin (ACT), antithrombin III (ATIII), and gelsolin by commercial enzyme-linked immunosorbent assay (ELISA) (Innotest, Innogenetics, Ghent, Belgium). For all biomarker measures, samples were continuously kept on ice, and assays were performed on sample aliquots after a single thaw from initial freezing.

Threshold Selection: The DIA software performs a log transformation of the volume ratios and uses them to generate a frequency histogram. A normal distribution is fitted to the main peak of the frequency histogram. After normalization, this fitted distribution curve centers on 0, which represents proteins with unaltered abundance, Model standard deviation (S.D.) is then derived based on the normalized model curve. 2 S.D., the volume ratio for 2S.D. based on the raw data, is the software-recommended cut-off. In a normally distributed data set, 95% of data points would fail within this value. Based on the observation that 2 S.D. ranged from 1.31 to 1.52 for the six individuals who were compared 2 weeks apart, gel features changing by >1.5 in spot volume were considered significant.

p Value Determination for Intraindividual Variation: The statistical significance of observing different levels of the same protein in multiple intraindividual comparisons was estimated by describing the data as a binomial distribution and calculating the probability of the observed events. Our null hypotheses are as follows; 1) all intra-individual comparison experiments are independent from each other, and 2) in any intra-individual comparison, protein levels should not change; therefore any observed change should be random and represent system fluctuation rather than a property of an individual protein. For any given experiment (intra-individual comparison) that follows the null hypotheses, the probability of any protein changing its expression level is pₒ. This value can be estimated by maximum a posteriori estimation; i.e. based on the observed number of protein spots detected in a given gel and the observed number of spots determined to have altered abundance (i.e. having a >1.5 spot volume ratio) between the two time points in an intraindividual comparison, we calculated pₒ, such that the probability of observing the experimental data given pₒ, is maximized. In N independent trials (in this case six intraindividual comparisons), the probability of observing the same protein having changed abundance in n or more individuals is as follows.

$$p ( i \geq n / N ) = {\sum }_{i = n … N} ( \begin{matrix}N \\ n\end{matrix} ) {{p}_{c}^{n} ( 1 - {p}_{c} )}^{N \cdot n} \qquad ( Eq . 1 )$$

pₒ, is determined to be 0.0140, and N is 6. Because not all of the “changed” spots are identified by MS/MS, this p value is likely an underestimation of the significance.

Hierarchical Clustering and Multidimensional Scaling Analysis: Hierarchical clustering was performed using Spotfire (Spotfire, Somerville, Mass.) software. Unweighted pair group method with arithmetic mean (UPGMA) was selected as the clustering method, and Euclidean distance was selected as the similarity measure. BRB Arraytools (linus.nci.nih.gov/BRB-ArrayTools.html) were used for multidimensional scaling analysis. Euclidean distance was used to measure similarity.

### Results:

Prior to comparing CSF samples between individuals to identify patterns of disease-associated proteins. It is important to examine variation within individuals over a short period of time so that one can better interpret potential changes in CSF between individuals, as well as changes within a given individual over a longer time span. In a first study, we analyzed 12 CSF samples, composed of pairs of samples from six individuals, obtained 2 weeks apart. Multiaffinity depletion, two-dimensional DIGE, and tandem mass spectrometry were used. A number of proteins whose abundance varied between the two time points were identified for each individual. Some of these proteins were commonly identified in multiple individuals. More importantly, despite the intraindividual variations, hierarchical clustering and multidimensional scaling analysis of the proteomic profiles revealed that two CSF samples from the same individual cluster the closest together and that the between-subject variability is much larger than the within-subject variability. Among the six subjects, comparison between the four cognitively normal and the two very mildly demented subjects also yielded some proteins that have been identified in previous AD biomarker studies.

One objective of this study was to evaluate variability in the CSF proteome associated with longitudinal collection of CSF from individuals. Utilizing single gel, multiple image analysis, we were able to identify within-subject differences with a high degree of confidence. By including a pooled sample in every gel as an internal standard, we were able to match and perform relative quantification of spots across gels and compare the degree of within-subject variation with that of between-subject variation. This study is an important component in the long term research program of the applicants to identify biomarkers for preclinical and very mild AD.

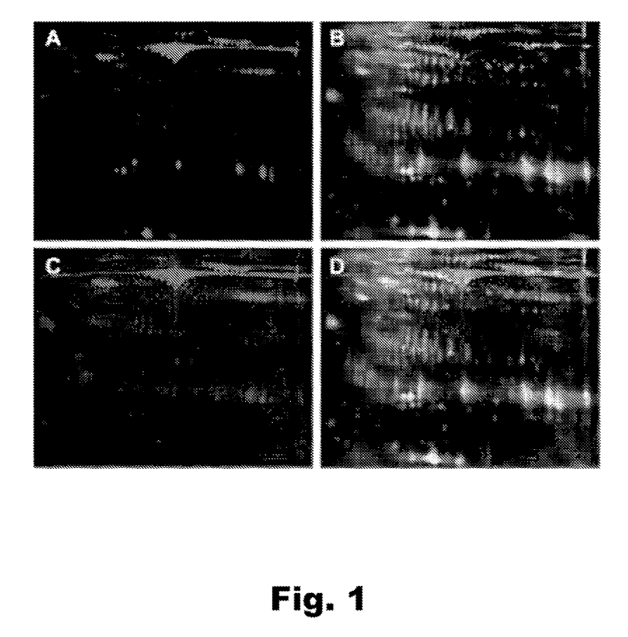

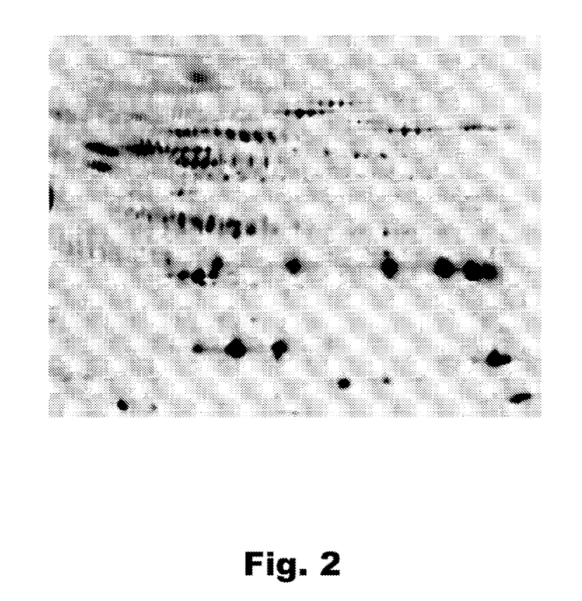

Because high abundance proteins comprise a large portion of the protein content of CSF and thus dominate 2-D gel images, we removed these proteins to increase our ability to detect and quantify proteins of lower abundance. We used a column-based multiple immunoaffinity system to remove six proteins (albumin, IgG, α₁-antitrypsin, IgA, transferrin, and haptoglobin) from human CSF. This depletion technique has been shown in a proteomic study on serum to be superior to three other similar depletion methods and resulted in a 76% increase in the number of protein spots defected (Chromy et al. (2004) J. Proteome Res. 3, 1120-1127). When loading the same amount of total protein, our CSF study showed a 99% increase in the number of spots detected (FIG. 1): up to —2,100spots were detected on a gel. FIG. 2 is a representative 2-D DIGE image of a postdepletion CSF sample used in this study.

Two CSF samples were taken from the same individual 2 weeks apart (time point 1 (T1) and time point 2 (T2)) and were analyzed on the same gel together with a pooled sample as an internal standard. Six individuals were analyzed, translating into 12 CSF samples and six gels. The internal standard was a pool that included an equal volume aliquot of all 12 CSF samples. Pairwise comparisons of individual spots were made between T1 and T2samplings for each individual to identify differences in protein abundance. A number of spots were selected for MS/MS analysis for each individual based on the following two criteria: 1) the fold change of the spot volume between the two time points was greater than 1.5 (see “Experimental Procedures” for threshold selection), and 2) the protein spot was well resolved and appeared as a symmetrical peak. A total of 104 spots met these criteria, ranging from eight to 34 spots per gel, and 73 of them were identified by MS/MS. Table I presents some of the identified proteins, along with the number of individuals sharing a greater than 1.5-fold change. The -fold change is the change in protein abundance at T2 compared to T1 (the direction of change was ignored).

TABLE 1
Proteins identified by MS/MS that vary in abundance
within individuals over a 2-week time period.

| Protein | GenBank ™ ID | Number | ~Fold change | p value |
|---|---|---|---|---|
| Transthyretin | 339685 | 6 | 1.92 ± 0.46 | 7.53 × 10⁻¹² |
| Chromogranin A | 180529 | 3 | 1.79 ± 0.09 | 5.32 × 10⁻⁸ |
| Complement component C4A | 443671 | 3 | 1.73 ± 0.31 | 5.32 × 10⁻⁵ |
| Hemopexin precursor | 386789 | 3 | 1.63 ± 0.09 | 5.32 × 10⁻⁵ |
| Osteopontin-a | 992948 | 3 | 1.50 ± 0.11 | 5.32 × 10⁻⁵ |
| β fibrinogen precursor | 182430 | 2 | 3.33 ± 1.21 | 0.00283 |
| Apolipoprotein E | 178849 | 2 | 3.13 ± 0.03 | 0.00283 |
| Complement component 3 precursor | 4557385 | 2 | 2.81 ± 1.60 | 0.00283 |
| Semaphorin L | 3551779 | 2 | 2.06 ± 0.74 | 0.00283 |
| Aprotinin | 58005 | 2 | 2.05 ± 0.51 | 0.00283 |
| Prostaglandin D₂ synthase | 54696706 | 2 | 1.97 ± 0.17 | 0.00283 |
| a₂-Macroglobulin | 177872 | 2 | 1.75 ± 0.18 | 0.00283 |
| Fibulin 1 precursor | 106018 | 2 | 1.64 ± 0.31 | 0.00283 |
| Chromogranin B precursor | 4502807 | 2 | 1.64 ± 0.12 | 0.00283 |
| Ubiquitin | 13569612 | 2 | 1.60 ± 0.06 | 0.00283 |
| Fibrinogen γ | 71828 | 1^{a} | 3.87 ± 2.55 | 0.0811 |
| Apolipoprotein H precursor | 4557327 | 1^{a} | 2.55 ± 0.30 | 0.0811 |
| PRO 1400 | 8650772 | 1^{a} | 1.99 ± 0.58 | 0.0811 |
| Transferrin | 31415705 | 1^{a} | 1.66 ± 0.18 | 0.0811 |
| Pyruvate kinase | 2117873 | 1 | 6.02 | 0.0811 |
| Complement C8-β propeptide | 29575 | 1 | 5.49 | 0.0811 |
| Angiotensinogen | 532198 | 1 | 4.72 | 0.0811 |
| Coagulation factor XII | 180359 | 1 | 2.15 | 0.0811 |
| Scrapie-responsive protein 1 | 37183198 | 1 | 1.62 | 0.0811 |
| Cerebroside sulfate activator protein | 337760 | 1 | 1.60 | 0.0811 |
| α albumin | 4501987 | 1 | 1.52 | 0.0811 |

^{a}Several isoforms were identified in the same individual

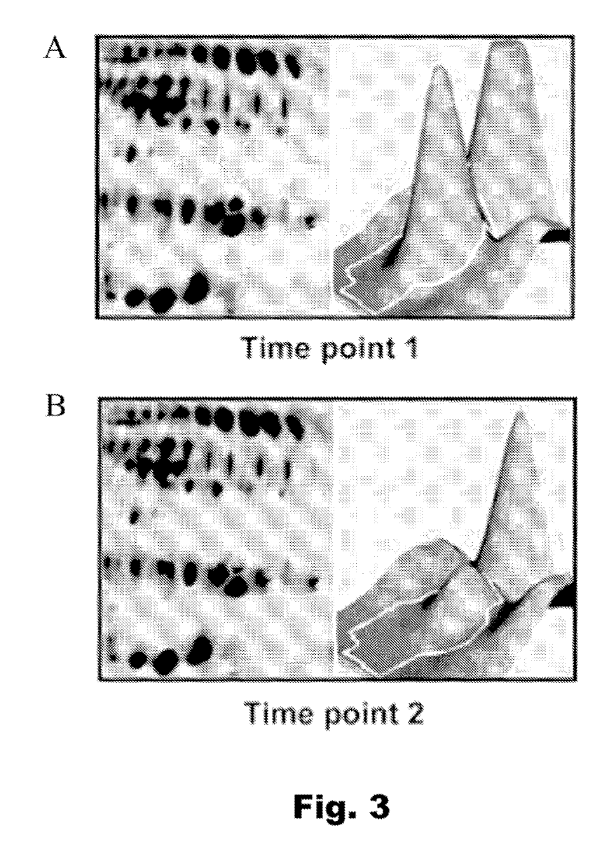

There was a certain degree of variability in the CSF proteome detected within an individual over a 2-week period. Representative gel images and 3-D representations of one of such proteins are shown in FIG. 3. There was a 3.1-fold change of the levels of this ApoE spot between the two time points. Although many protein constituents in the CSF are derived from plasma, some of these changed proteins (Table 1) have been shown to be enriched in CSF or derived from nervous tissue, such as prostaglandin D₂ synthase, transthyretin, apolipoprotein E (apoE), chromogranin A, chromogranin B, semaphorin L, and scrapie-responsive protein 1. Interestingly a number of the same proteins are found to vary in multiple individuals. We calculated the p value of a given protein appearing as changed by chance in one or more individuals (Table 1) and found that it is statistically significant when a protein is shown as changed in two or more individuals (using a cut-off p value of 0.05). The protein found to vary most often is transthyretin (six of six individuals). Other commonly variable (isoforms of) proteins include chromogranins A and B, complement component 3 and C4A, hemopexin precursor, osteopontin a, β fibrinogen precursor, apoE, semaphorin L, prostaglandin D₂ synthase, α₂-macroglobulin, fibulin precursor, and ubiquitin. Aprotinin, a synthetic peptide present in the lysis buffer, appeared as a changed protein. Transthyretin and apoE have been shown to promote the solubility, transport, and clearance of amyloid-β (Aβ), a molecule important in the pathogenesis of AD. For transthyretin, multiple isoforms were found to vary within individuals (in the same direction for a given individual), whereas only one isoform of apoE was found to vary. In fact, our apoE ELISA data showed that total apoE level does not vary significantly within individuals. The direction of the intra-individual changes (increase versus decrease when comparing the initial and subsequent CSF sampling) did not show any trends among individuals. These findings strongly suggest that intra-individual variations in the CSF proteome reflect the dynamic steady states of those proteins. These data also suggest that the levels of some (isoforms of) proteins in CSF tend to fluctuate more significantly than others due to the nature of their metabolism as opposed to standard errors in experiments. Such intra-individual protein abundance variations are a caveat when considering these proteins as potential disease-related biomarkers.

An important goal of this study was to quantify the degree of intra-individual variation in relation to variation between individuals. For that purpose, protein spots were matched across all six gels so that a proteomic profile containing matched spots can be created for each sample, allowing for statistical comparisons to be made between individual profiles.

One advantage of 2-D DIGE is the ability to perform intergel matching and comparison through the inclusion of an internal standard on each gel. Nevertheless the assumption in using 2-D gel image analysis to measure relative protein concentration is that matched spots (spots that are located at the same position in different gels) correspond to the identical protein. This assumption was tested by first matching all six gels using one of the pooled samples (internal standard) as a master image, from which 306 protein spots across all six gels were matched. Sixteen matched spots that were well resolved and distributed across the entire gel were selected and analyzed with MS/MS. For 14 spots, protein identifications were obtained from more than two gels. For 11 of the 14 spots, the identified proteins were the same. Examination of the 3-D gel images revealed that, for the three spots that gave different proteins there was evidence (e.g. a ridge) of another protein underneath the most prominent gel feature or matching/picking aberrations. Our conclusion is that matched, well-resolved gel features correspond to the same protein.

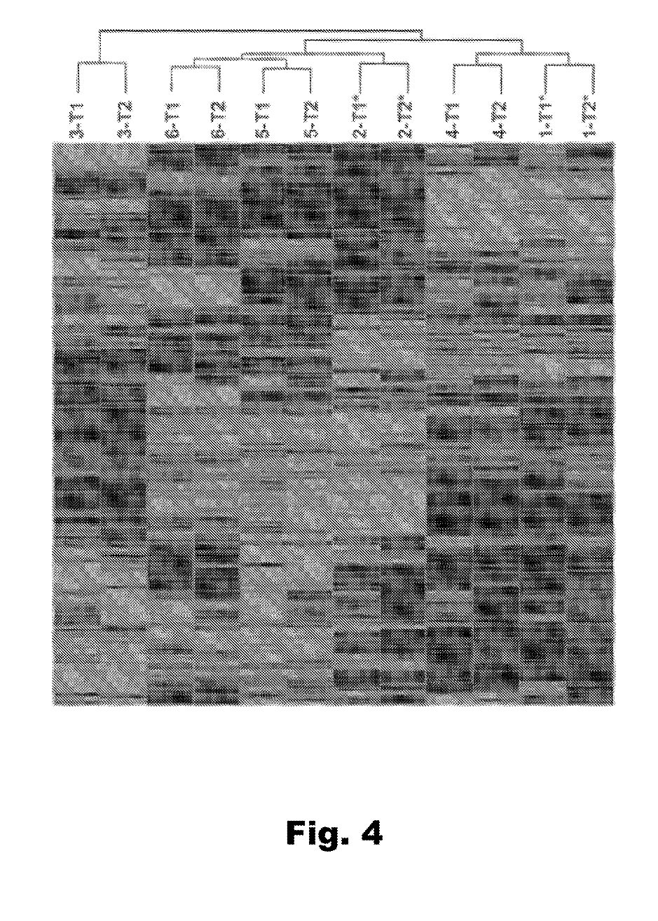

After intergel matching, a proteomic profile that consisted of standard abundance (i.e. -fold change in protein abundance compared with the pooled internal standard) was generated for matched spots for each of the 12 CSF samples. Because standard abundance is derived using the spot volume of the pooled sample as a denominator, it can be considered as the relative abundance of a protein spot. In this dataset, two statistical analyses were applied to assess the relationship among the CSF samples. First, hierarchical clustering analysis was performed on the proteomic profiles. Hierarchical clustering orders objects in a treelike structure based on similarity (i.e. in this case, “pattern similarity”). Clustering analysis is used extensively in the mining of gene expression data generated by functional genomic studies, but its application to protein expression data remains limited. Through the inclusion of an internal standard, a dataset was created that was very similar to datasets derived from GeneChip or microarray experiments and therefore allowed for the application of clustering algorithms to 2-D gel image analysis. The results, including a dendrogram and a heat map, are presented in FIG. 4. The dendrogram reveals that each pair of intraindividual CSF samples (T1 and T2) was clustered the closest together. As can be visualized in the heat map, the proteomic profiles of infra-individual samples are most similar to each other and distinctively different from other individuals profiles. The two CDR subjects with very mild AD (i.e. CDR 0.5, marked with an asterisk) did not cluster the closest together, indicating that there were no obvious global changes of protein expression between the CDR 0.5 and CDR 0 samples in this initial study. To rule out the possibility that the two samplings from the same subject cluster together simply because they were run on the same gel, we analyzed T1 samples from two subjects on one gel and T2samplings from these two subjects on another gel. Clustering analysis of the proteomic profiles showed that longitudinal samples from the same subject still cluster the closest together. This further demonstrates the validity of the intergel comparison.

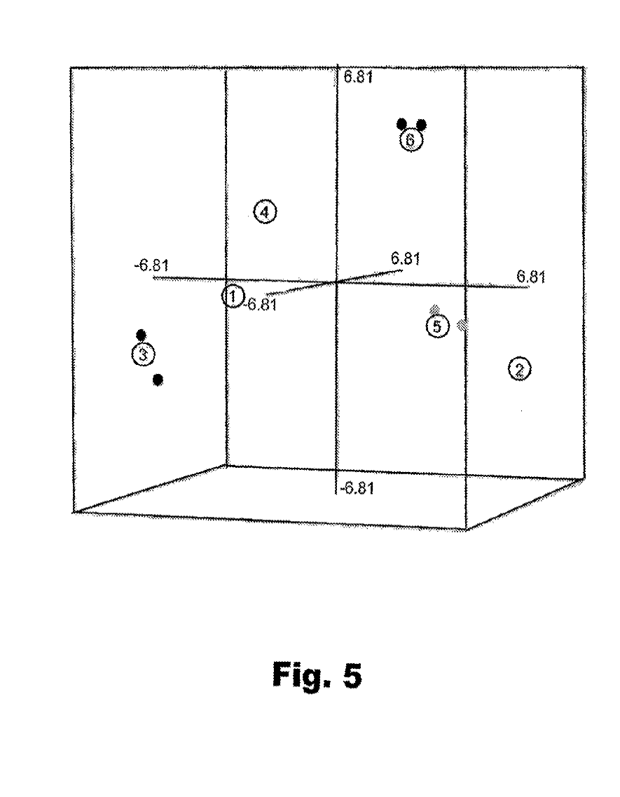

Second, multidimensional scaling analysis was performed on the proteomic profiles. Like hierarchical clustering, multidimensional scaling is commonly used in microarray research as an analysis tool. Its purpose is to capture as much variation in the data as possible in a minimal number of dimensions (two or three) so that trends in data are more obvious. In effect, one is attempting to reduce the dimensionality of the data to summarize the most influential (i.e. defining) components while simultaneously filtering out noise. The distance between two samples can be thought to represent the similarity between them; the smaller the distance, the more similar the samples. FIG. 5 is a 2-D projection of the 3-D scatter plot of the proteomes of the 12 CSF samples. CSF samples from the same subject are represented with the same color. As can be observed from the graph, there is a short distance between two samples from the same individual, and this distance varies among individuals. However, the distance between intraindividual CSF samples is much smaller than between samples from different individuals. These data highlight that intraindividual variation in the CSF proteome is much smaller than the interindividual variation,

Because our sample set included four CDR 0 subjects (eight CSF samples) and two CDR 0.5 subjects (four CSF samples), we were able to evaluate possible CDR group-associated differences. As mentioned earlier, 306 spots were matched across six gels/12 individual CSF samples. For each spot in a given CSF sample, there is a corresponding standard abundance (representing the relative abundance of this protein spot) that can be compared across the 12 samples. Therefore, we were able to perform a t test (in the BVA module of the DeCyder software) on the matched spots by designating the eight CDR 0 CSF samples as group 1 and the four CDR 0.5 samples as group 2. We selected the top ranking (p<0.02) and well resolved spots and subjected them to MS/MS analysis. Eleven of the 13 spots selected were found to represent eight different proteins (Table 2). Interestingly four of these proteins have been shown to have altered levels in AD CSF, including α₁β-glycoprotein, prostaglandin D₂ synthase, cystatin C, and β₂-microglobulin. Consistent with previous studies, α₁β-glycoprotein is decreased in the CDR 0.5 group, whereas cystatin C and β₂-microglobulin are increased. Prostaglandin D₂ synthase was found to increase in the CDR 0.5 group, which is consistent with a previous study, whereas another study reported a decrease of prostaglandin D₂ synthase. Several isoforms of chitinase 3-like 1, also known as GP-39 cartilage protein, were found to be increased in the CDR 0.5 group. This protein is primarily produced by human chondrocytes and synovial fibroblasts and has been shown to be a target antigen in patients with rheumatoid arthritis.

TABLE 2
CSF proteins found to be differentially expressed
between CDR 0.5 and CDR 0 groups.

| Spot | | GenBank ™ | | Direction |
|---|---|---|---|---|
| ID | Protein | ID | p value | of change |
| 1 | α₁β-Glycoprotein | 69990 | 0.011 | Decrease |
| | Complement component 3 | 4557385 | 0.011 | Decrease |
| | precursor | | | |
| 2 | Apolipoprotein H precursor | 4557327 | 0.0071 | Decrease |
| 3 | Chitinase 3-like 1 | 4557018 | 0.011 | Increase |
| 4 | Chitinase 3-like 1 | 4557018 | 0.014 | Increase |
| 5 | Prostaglandin D₂ synthase | 55962672 | 0.015 | Increase |
| | Chitinase 3-like 1 | 4557018 | 0.015 | Increase |
| 6 | Prostaglandin D₂ synthase | 54696706 | 0.00022 | Increase |
| 7 | Prostaglandin D₂ synthase | 54696708 | 0.016 | Increase |
| 8 | Prostaglandin D₂ synthase | 54696706 | 0.011 | Increase |
| 9 | Cystatin C | 181387 | 0.019 | Increase |
| 10 | Thioredoxin | 231098 | 0.0058 | Increase |
| 11 | β₂-Microglobulin | 34616 | 0.0022 | Increase |

Although this sample size was very small, five of eight differentially expressed proteins identified by MS/MS have been implicated in previous AD biomarker studies, and for three of them, these results (increase versus decrease) are consistent with previous reports. Importantly only one of these proteins (i.e. prostaglandin D₂ synthase) was identified to display intra-individual variation. These preliminary differences that were found need to be validated with a much larger sample set; however, these results suggest that, given appropriate sample selection, protein quantification, and profile comparison, one may not require a large set of samples to identify disease-related biomarkers.

### Example 4

### Materials and Methods for Example 4

Subjects: Research subjects were participants at the Alzheimer's Disease Research Center (ADRC) at the Washington University School of Medicine (WUSM) and were recruited by the ADRC to this study. All subjects gave informed consent to participate in this study and all protocols were approved by the institutional review board for human studies at Washington University. Study investigators were blind to the cognitive status of the participants, which was determined by ADRC clinicians in accordance with standard protocols and criteria, as described previously (Berg et al,. Arch Neurol (1998) 55:326-335; Morris et al., Ann Neurol (1988) 24:17-22). Subjects were assessed on clinical grounds to be cognitively normal in accordance with a Clinical Dementia Rating (CDR) of 0 (n=55) or to have very mild (CDR 0.5; n=20) or mild (CDR 1; n=19) dementia that is believed to be caused by AD as described (Morris et al., Arch Neurol (2001) 58:397-405).

CSF and plasma collection: CSF (20-35 ml) was collected at 8:00 AM after overnight fasting, as described previously (Fagan et al., Ann Neurol (2000) 48:201-210). Lumbar punctures (LPs) (L4/L5) were performed by a trained neurologist using a 22-gauge Sprotte spinal needle. CSF samples were free from any blood contamination. Samples were gently inverted to avoid gradient effects, briefly centrifuged at low speed to pellet any cellular elements, and aliquoted (500 μl) into polypropylene tubes before freezing at −80° C. Fasted blood (10-15 ml) was also obtained from each subject just before LP, and plasma was prepared by standard centrifugation techniques. Plasma samples were aliquoted (500 μl) info polypropylene tubes before freezing at −80° C.

Multi-affinity immunodepletion of abundant CSF proteins: Since albumin, IgG, α1-antitrypsin, IgA, transferrin and haptoglobin collectively account for ˜80% total CSF protein content, they were selectively removed in order to enrich for proteins of lower abundance. An antibody-based multi-affinity removal system (Agilent Technologies, Palo Alto, Calif.) was employed according to the manufacturer's instructions, as described in the materials and methods for examples 1-3.

2D-DIGE: Twelve CSF samples were analyzed by 2D-DIGE: CDR 1 subjects (n=6) and age-matched CDR Q controls (n=6). These samples were selected based on Aβ42 concentration, i.e. CDR 1 samples were selected whose Aβ42 concentration is less than 500 pg/ml and the converse for CDR 0samples. Fifty μg of protein from each depleted CSF sample was labeled with 400 pmol of one of three N-hydroxsuccinimide cyanine dyes for proteins (GE Healthcare, Piscataway, N.J.), mixed with 30 μg of unlabled protein from the same sample, diluted with rehydration buffer, combined according to experimental design, and equilibrated with immobilized pH gradient (IPG) strips (24 cm; pH 3-10, nonlinear). The three samples that were equilibrated with each IPG strip consisted of a CDR 0 sample, a CDR 1 sample and a pooled sample (pooled using an equal volume aliquot of each of the 12 CSF samples) (labeled with Cy3) as the internal standard. To avoid possible dye-related bias, half of the CDR 0samples were labeled with Cy2 and half with Cy5 and the same protocol was used to label the CDR 1 samples. First-dimension isoelectric focusing was performed at 65.6 kV-h in an Ettan SPGphor system (GE Healthcare). The second dimension was performed on a gradient SDS-PAGE gel (10-20%). The Cy2, Cy3. Cy5-labeled images were acquired on a Typhoon 9400 scanner (GE Healthcare).

Image analysis: Intra-gel spot detection, quantification and inter-gel matching and quantification were performed using Differential in-gel Analysis (DIA) and Biological Variation Analysis (BVA) modules of DeCyder software v5.01 (GE Healthcare), respectively as described previously (Hu et al,, Mol Cell Propteomics (2005) 4:2000-9, hereby incorporated by reference in its entirety). A subset of protein spots (n=514) were matched across all six gels. Student's t-test was performed to compare the average (relative) abundance of a given spot between the two groups (CDR 1 vs, CDR 0). Spots that had a p value below 0.05 and were well resolved were selected for subsequent mass spectrometry analysis.

Protein digestion and mass spectrometry: Gel spots of interest were flagged with the DeCyder software and the X and Y coordinates were used by a robotic spot picker (ProPic; Genomics Solutions, Ann Arbor, Mich.) to cut and transfer gel features into a 96 well plate for in situ gel digestion with trypsin using a modification of a previously described method (Havlis et al. Anal Chen (2003) 75:1300-6). Briefly, excised gel cylinders (2 mm in diameter) were robotically transferred to 96 well plates (Axygen Scientific, Union City, Calif.) and over-layered with 100 μL of water. After washing and trypsin digestion (Sigma, St. Louis, Mo.) in 5 μL of 5 mM tri-ethyl-ammonium bicarbonate buffer, (pH 8.0; Sigma), an aliquot (0.5 μL) was removed from each well and placed in a microfuge tube (200 μL) containing 0.5 μL of MALDI Matrix (Agilent Technologies). The tubes were vortexed, spun in a microfuge for 30 s and spotted (1 μL) onto a stainless steel target (192 spot plate) for MALDI-TOF/TOF mass spectrometry as previously described (Bredemeyer et al., PNAS (2004) 101:11785-90), To the remaining gel samples, 12 μl of aqueous acetonitrile/formic acid (1%/1% (Sigma) was added and the plate was incubated at 37° C. for 1 h. The samples (˜10 μL) were then transferred to polypropylene auto sampler vials, spun at 10,000×g for 15 min in a Sorvall centrifuge equipped with a HB-6 rotor, and stored at −80° C. for analysis by LC-MS/MS. LC-MS/MS was performed using a capillary LC (Eksigent, Livermore, Calif.) interfaced to a nano-LC-linear quadrupole ion trap Fourier transform ion cyclotron resonance mass spectrometer (nano-LC-FTMS) (King et al., Anal Chem (2006) 78:2171-81).

Database searching was performed using MASCOT 1.9.05software (Matrix Science, Oxford, UK) against the NCBI non-redundant database (Mar. 24, 2005). Protein identifications were supported by at least two peptides with Mascot scores of >40 and accurate measurements of the peptide parent masses within 5 ppm. Protein identifications were confirmed by manual interpretation of the fragmentation spectra with a minimal acceptance criterion of four contiguous b or y ions for each peptide sequence.

ELISA analyses: Aβ42 and total tau concentrations in the CSF samples were analyzed by a commercially available enzyme-linked immunosorbent assay (Innotest; Innogenetics, Ghent, Belgium). Albumin ELISA was performed with an ELISA kit (Bethyl Laboratories, Montgomery, Tex.). A sandwich ELISA was developed for ACT measurement; rabbit anti-human ACT antibody (1:1000; DAKO, Carpinteria, Calif.) was used for capture and a sheep anti-human ACT antibody (15000; The binding site, San Diego, Calif.) was used for detection. ACT purified from human plasma was used as standard (Sigma). For ATIII measurement a sandwich ELISA was developed; rabbit anti-human ATIII antibody (1:1000; DAKO) was used for capture and a mouse monoclonal anti-human ATIII (clone HYB 230-04, 1:5000; Assay designs, Ann Arbor, Mich.) was used for detection. ATIII purified from human plasma was used as standard (Sigma). For ZAG, a sandwich ELISA was developed; rabbit anti-human ZAG antibody (1:1000; gift from Dr. Iwao Ohkubo, Shiga University of Medical Science, Japan) was used for capture and a mouse monoclonal anti-human ZAG antibody (clone 1D4, 1:100; Santa Cruz Biotechnology, Santa Cruz, Calif.) was used for detection, ZAG purified from human seminal plasma was used as standard (gift from Dr. Iwao Ohkubo). For gelsolin ELISA, CSF samples were directly coated on the plate, followed by incubation with a mouse monoclonal anti-gelsolin antibody (clone GS-2C4, 1:2000; Sigma). Gelsolin purified from human plasma was used as standard (Sigma). For AGT measurement, a sandwich ELISA was used: mouse monoclonal anti-AGT antibody (clone F8A2, 1:285; gift from Dr. Claus Oxvig, University of Aarhus, Denmark) was used for capture and a chicken antibody (1:1200; gift from Dr. Claus Oxvig) was used for detection. AGT purified from human plasma was used as standard (Calbiochem, San Diego, Calif.). For CNDP1 ELISA, CSF samples were directly coated on the plate, followed by incubation with goat anti-human CNDPI antibody (1:500; R&D systems, Minneapolis, Minn.).

For all ELISA measurements: 1) Biotinylated secondary antibody (1:5000; Jackson ImmunoResearch, West grove, Pa.) and poly-HRP streptavidin (1:2000; Research Diagnostics, Concord, Mass.) were used; for color development, TMB super slow or TMB super sensitive (Sigma) were used; 2) Samples were kept continuously on ice, and assays were performed on sample aliquots after a second thaw after initial freezing; 3) The levels of a given protein in all CSF samples were measured in the same experiment; 4)Data from a single experiment is presented and similar data have been obtained from at least one replicate experiment.

Statistical analyses: The Shapiro-Wilk test was used to test the hypothesis of a normal distribution for levels of ACT, ATIII, ZAG, CNDP1, Aβ42, and tau. The distributions were log transformed to approximate a normal distribution. Values were then adjusted for age at lumbar puncture, gender, and the number of APOE4 alleles. The residuals were tested for association with case-control status using a t-test.

The area under the receiver operating characteristic curve (AUG) for each trait was calculated using the ROCR package in R (http://www.R-project.org: (Sing et al, Bioinformatics (2005) 21:3940-41)). A method proposed by Xiong et al (Med Decis Making (2004) 24:659-89) was implemented to determine the optimum linear combination of these traits and calculate confidence Intervals on the AUG. This method requires a complete dataset; removal of individuals with missing phenotype data resulted in a slight decrease in the sample size (N=83).

Immunohistochemistry: AGT immunostaining was performed on ethanol fixed paraffin sections (5 μm thickness) of human brain (frontal cortex) with a rabbit anti-human ACT antibody (1:100; Accurate chemical Westbury, N.Y.). ATIII immunostaining was performed on formalin fixed frozen sections (12 μm thickness) of human brain (frontal cortex) using a rabbit anti-human ATIII antibody (1:500; Dako). The staining was developed chromogenically (DAB). Standard immunohistochemical protocol was employed for the above immunostaining (Han et al., Neurobiol Dis (200) 7:38-53). Thioflavine-S and X-34 staining was performed as described (Bales et al., Nature Genetics (1997) 17:263).

### Results:

Sample selection: The CSF proteomes of 6 mild AD subjects were compared to those of 6 age-matched cognitively normal subjects. The mild AD subjects were clinically characterized with a CDR score of 1, indicating mild dementia and the controls all had a CDR score of 0, indicating non-demented. To maximize the likelihood of selecting CDR 1 subjects who truly had AD pathology from CDR 0 subjects who did not, selection was based on CSF Aβ42 concentrations. A recent study comparing in vivo amyloid imaging with CSF levels of various biomarkers showed that Aβ42 is an excellent marker of cerebral amyloid deposition, independent of clinical diagnosis Fagan et al., Ann Neurol (2006) 59:512-19). The CSF samples were analyzed individually (as opposed to pooling the samples) to increase the statistical power of the analysis.

Candidate biomarkers identified. Upon 2D-DIGE analysis, 514 protein spots were matched across all six gels. Since a pooled sample was included on each gel, the relative abundance of the matched spots were normalized in all 12 individual CSF samples. Student's t-test (using p<0.05 to define statistical significance) was performed to examine whether the average (relative) abundance of a given protein spot in the two CDR groups was significantly different. Twenty-one spots were found that displayed differential abundance (p values <0.05) between the two groups. Three spots were excluded due to poor gel resolution and the remaining 18 were selected for excision, in gel tryptic digestion, and mass spectrometry identification.

To estimate whether most of these proteins are truly different in abundance as opposed to artifacts, the false discovery rate (FDR) was assessed in this study by using data from our previous study with this method (see examples 1-3) to approximate a “same-same comparison”, in that study, the same comparative proteomic approach (depletion—followed by 2D-DSGE—MS/MS) was applied to the analysis of 12 CSF samples, composed of pairs of samples from the same six individuals, obtained 2 weeks apart. When the 6 CSF samples (as a group) obtained at time point 1 were compared to the 6 samples (as a group) obtained at time point 2, it is similar to a ‘same-same comparison’ because CSF samples from the same subject should be quite similar. When that comparison was performed, 0 spots were found (out of 306 matched spots) that were differentially expressed between the two groups (using a p value of 0.05 as cutoff). This result indicates that the false positive rate in the study (when using a p value of 0.05 as cut-off) is likely to be very low.

Protein identifications of the 18 spots are shown in Table 3. The protein identity of 16 of the 18 spots were successfully determined. For some spots, multiple proteins have been identified within one spot, most likely due to comigration of certain proteins. In addition, some proteins were identified in multiple proteins spots, including α1-antichymotrypsin (ACT) and antithrombin III (ATIII), likely reflecting the existence of different protein isoforms (e.g., post-translational modifications). A total of 11 proteins were identified as candidate biomarkers for mild AD. Levels of gelsolin (GSN) and carnosinase I (CNDP1) were found to be lower in the mild AD group compared to the CDR 0 group whereas levels of the other candidate markers were higher in the CDR 1 group compared to controls. The relative difference in abundance between the averages of the two groups observed in 2D-DIGE is moderate, most within ±2 fold.

TABLE 3
List of proteins that were identified through 2D-DIGE
and mass spectrometry analysis to have differential
abundance in mild AD and control CSF samples.

| | | Change in AD in | |
|---|---|---|---|
| | | the CDR 1 group | |
| | | compared to the | P value |
| Spot | Protein | CDR 0 group | (student's t-test) |
| 1 | Antithrombin III | increased | 0.028 |
| 2 | Antithrombin III | increased | 0.023 |
| 3 | Antithrombin III | increased | 0.033 |
| | Angiotensinogen | | |
| 4 | α-1-antichymotrypsin | increased | 0.031 |
| 5 | Camosinase I | decreased | 0.018 |
| | Secretogranin III | | |
| | Kininogen | | |
| 8 | α-1-antichymotrypsin | increased | 0.031 |
| 7 | Antithrombin III | increased | 0.022 |
| 8 | α-1-antichymotrypsin | increased | 0.0091 |
| 9 | No ID | increased | 0.0041 |
| 10 | α-1-antichymotrypsin | increased | 0.015 |
| 11 | α-1-antichymotrypsin | increased | 0.011 |
| | Secretogranin III | | |
| 12 | α-1-antichymotrypsin | increased | 0.0013 |
| | Secretogranin III | | |
| | Kininogen | | |
| 13 | Angiotensinogen | increased | 0.005 |
| | Kinongen | | |
| 14 | Zinc α2-glycoprotein | increased | 0.027 |
| 15 | Zinc α2-glycoprotein | increased | 0.014 |
| | Chromagranin B | | |
| 16 | Gelsolin | decreased | 0.028 |
| 17 | No ID | decreased | 0.048 |
| 18 | Neuronal pentraxin | decreased | 0.043 |
| | Prostaglandin D2 synthase | | |
| | β trace | | |
| | Secretogranin III | | |

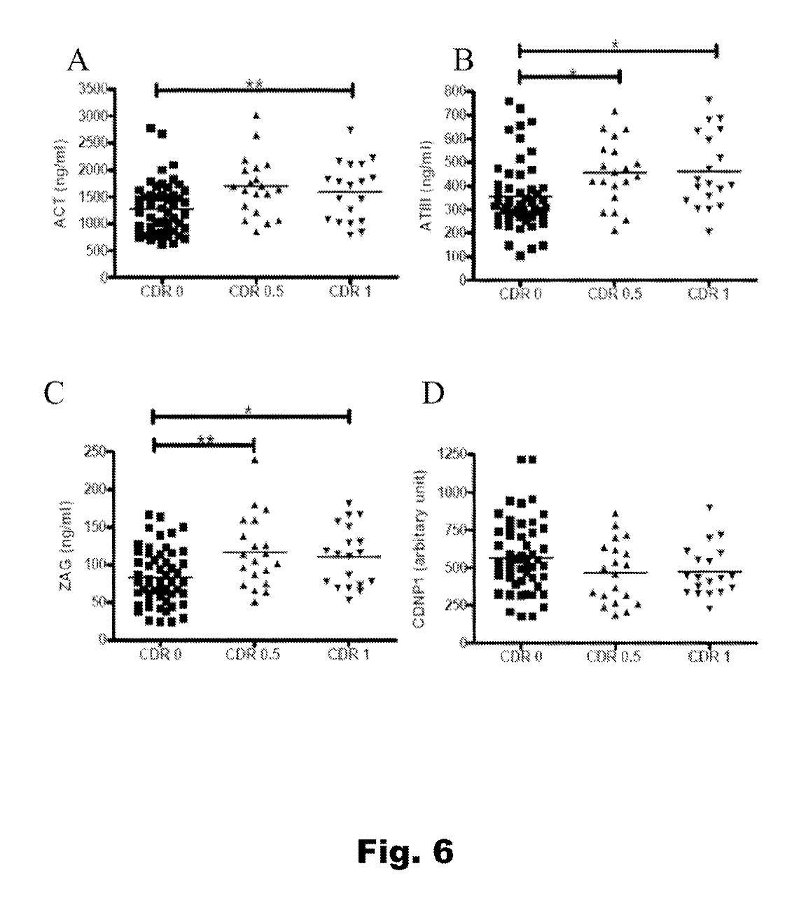

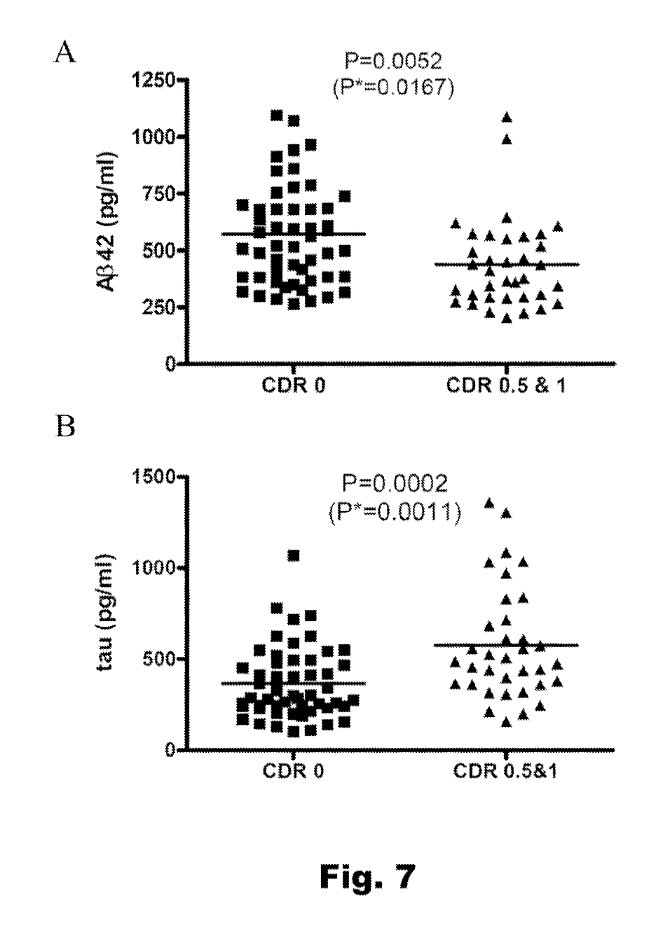

Validation of candidate biomarkers with ELISA. In order to confirm the findings with another method and also to be more quantitative, the levels of candidate biomarkers were first assayed in the original 12 CSF samples (CDR 0 (n=6), CDR1 (n=6)) using ELISA. Based on the availability of antibodies, the following candidates were assessed: ACT, ATIII, Zinc-α2-glycoprotein (ZAG), CNDP1, GSN and angiotensinogen (AGT). Then, a larger test set of CSF samples of cognitively normal subjects (CDR 0, n=49) were assayed versus subjects with very mild (CDR 0.5, n=19) or mild dementia (CDR 1, n=13) judged to be due to AD. The goal was to determine whether the findings could be validated in a larger, independent sample set. This level of validation is very important and has not been done before in previous AD proteomic biomarker studies to our knowledge. For all the candidate biomarkers evaluated, when the larger sample set was assayed, the same trend was observed as when the 12 original samples were assayed. Therefore, values from the 12 original samples were combined with the test sample set in the subsequent graphs. In addition, since no differences between the CDR 0.5 and CDR 1 groups were observed, these groups were combined to form the mild AD group and were then compared to the CDR 0 group. ELISA data that display CDR 0.5 and CDR 1 separately can be found in FIG. 6. For reference, the concentrations of Aβ42 and total tau (which have been obtained previously to this study) in these CSF samples were included (FIG. 7).

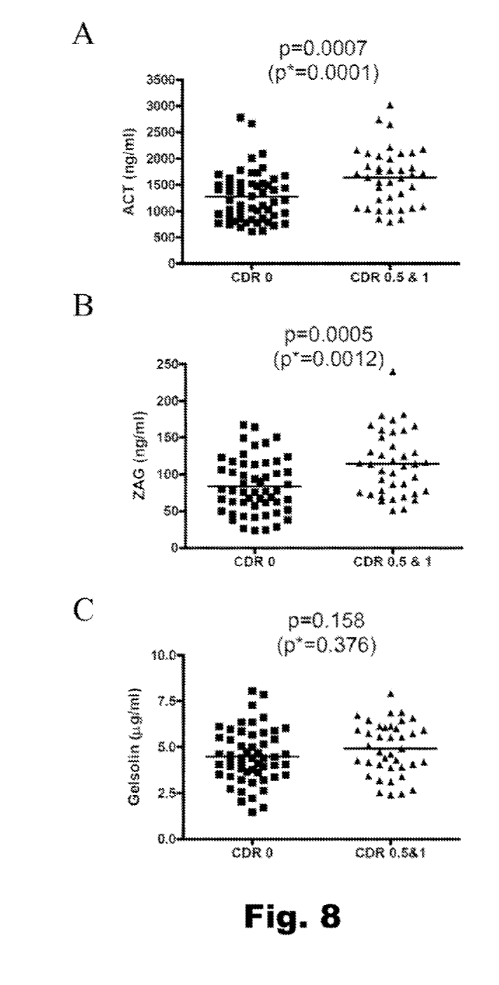

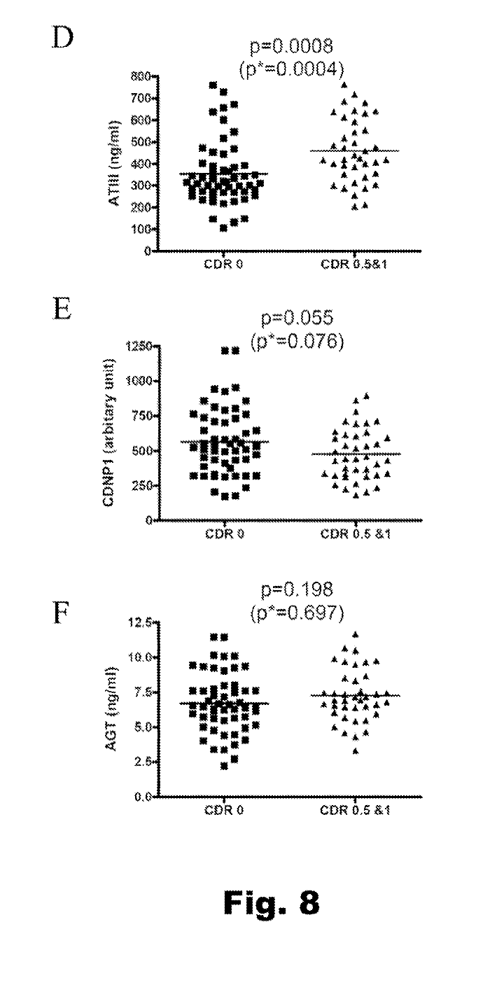

As shown in FIG. 8, the CSF levels of ACT, ATIII and ZAG are significantly increased in the very mild/mild AD group, confirming the 2D-DIGE findings. There is a trend for a decrease in CNDP1 levels in the very mild/mild AD group (FIG. 8), though this was not quite statistically significant (p=0.055). In contrast to the 2D-DIGE findings, a difference in levels of GSN or AGT between the two groups was not confirmed (FIG. 8). After log transformation (to approximate a normal distribution) the datasets were examined for any possible interactions between CSF concentrations of candidate biomarkers (including Aβ42 and total tau) and age/gender/number of APOE4 alleles. A significant correlation was found between the number of APOE4 alleles and CSF levels of Aβ42, total tau and AGT. There is also a positive correlation between age and CSF levels of ACT and GSN. In addition, an interaction between gender and CSF ZAG concentrations was observed, with males having higher levels than females. The initial observations based on the raw concentration data are not qualitatively different from those using the log-transformed and adjusted traits. The adjusted p values were included in FIG. 8 (designated by p*) for comparison. Since the differences in levels of GSN or AGT between the two groups were not confirmed, these two candidates were not included in subsequent analyses.

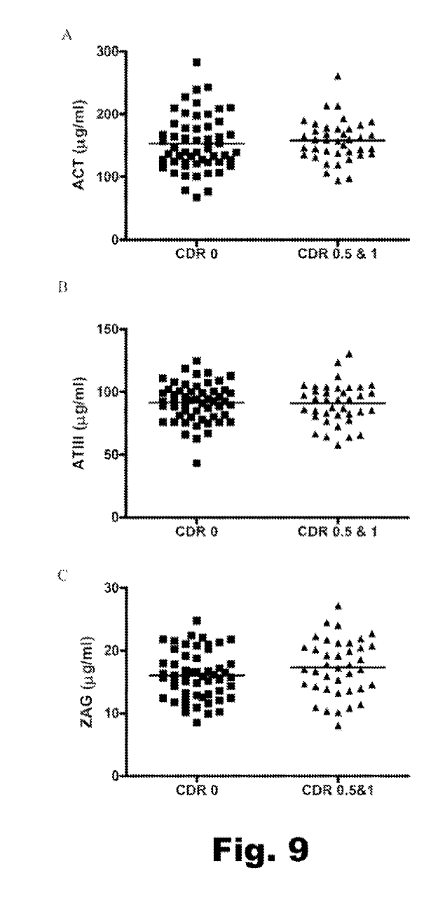

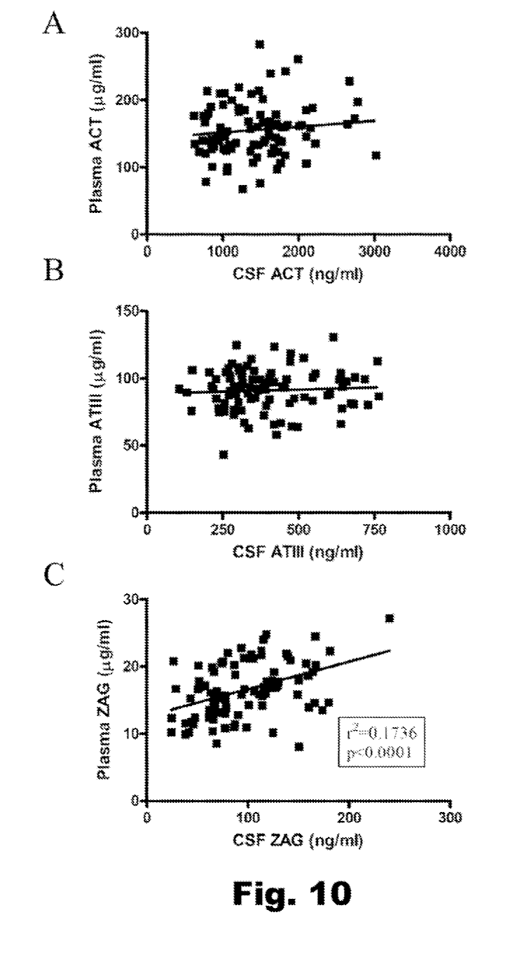

Next, whether similar differences exist in plasma as were found in CSF was assessed. Levels of ACT, ATIII and ZAG in paired plasma samples were not significantly different between the very mild/mild AD and control groups (FIG. 9). There was also no significant correlation between the CSF and plasma levels of ACT or ATIII, but there was a weak correlation between the CSF and plasma levels of ZAG (FIG. 10).

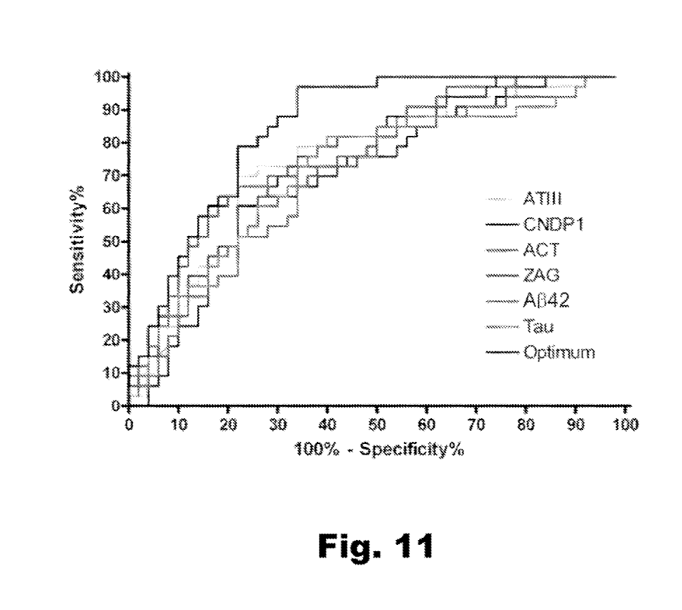

The relationships among the CSF concentrations of the candidate biomarkers (together with Aβ42 and tau) were examined (Table 4). None of the newly identified candidate biomarkers correlate with Aβ42 though ACT, CNDP1 and ZAG moderately correlate with tau. interestingly, there are very strong, highly significant correlations between ACT, ATIII and ZAG (but not CNDP1) (Table 4). To preliminarily assess the potential use of these candidate biomarkers in differentiating subjects with very mild/mild AD from controls, a method proposed by Xiong et al. (Med Decis Making (2004) 24:659-69) was applied to determine the optimum linear combination of these biomarkers and calculated the confidence intervals on the AUC (the area under the receiver operating characteristic curve). Individual candidates by themselves are very comparable to those of Aβ42 and total tau in differentiating very mild/mild AD from controls (Table 5, FIG. 11). importantly, when all biomarkers are optimally combined, this results in a higher AUG and sensitivity than for any single biomarker.

TABLE 4
Pearson Correlation Coefficients for the normalized, adjusted biomarkers.

| | ATIII | CNDP1 | ACT | ZAG | Aβ42 | TAU |
|---|---|---|---|---|---|---|
| ATIII | 1 | | | | | |
| CNDP1 | −0.007 (0.95) | 1 | | | | |
| ACT | 0.73 (<.0001) | 0.030 (0.78) | 1 | | | |
| ZAG | 0.66 (<.0001) | 0.026 (0.81) | 0.71 (<.0001) | 1 | | |
| Aβ42 | 0.018 (0.87) | 0.090 (0.40) | −0.056 (0.61) | 0.024 (0.83) | | |
| TAU | 0.092 (0.40) | 0.32 (0.003) | 0.36 (0.0009) | 0.32 (0.0024) | −0.065 (0.55) | 1 |

Coefficients were derived using log-transformed data that were also adjusted for interacting factors (i.e. age, gender or the number of APOE4 allele). P-values are displayed in the parentheses.

TABLE 5
Comparison of the AUC of the optimum linear combination
(Optimum) to that of each individual biomarker (AUC:
area under the receiver operating curve).

| | | Sensitivity at | |
|---|---|---|---|
| Marker | AUC (SE) | 80% Specificity | p-value vs. Optumim |
| ATIII | 0.75 (0.10) | 0.48 | 0.29 |
| CNDP1 | 0.65 (0.12) | 0.24 | 0.099 |
| ACT | 0.75 (0.11) | 0.3 | 0.32 |
| ZAG | 0.72 (0.10) | 0.42 | 0.21 |
| Aβ42 | 0.64 (0.12) | 0.36 | 0.085 |
| tau | 0.68 (0.11) | 0.38 | 0.14 |
| Optimum | 0.88 (0.07) | 0.7 | — |

Cellular localization of ACT and ATIII. The cellular localization of some of the candidate biomarkers were examined in human and transgenic AD mouse brain. Consistent with previous reports (Abraham et al., 1998; Abraham et al., Neurobiol aging et al. (1990) 11:123-29), ACT is co-localized with amyloid plaques in human AD brain and is also present in neurons and astrocytes. Also, antibodies to ATIII label amyloid plaques and neurofibrillary tangles (NFT) in human AD brain, in accordance with a previous report (Kalatira et al, Am J Pathol (1993) 143:886-893).

In this study, candidate biomarkers for mild AD were identified by analyzing a small group of CSF samples (mild AD vs. control) with a comparative proteomic approach. Importantly, selected biomarkers were validated with ELISA using a larger sample set. Among the identified biomarkers is ACT, which has been identified previously in other reports. Novel biomarkers such as ATIII, ZAG and CNDP1 were also identified, whose roles as AD biomarkers in CSF have not been previously assessed and validated. This is the first proteomic study on AD biomarkers that not only discovered candidate protein biomarkers, but also validated these candidates in a much larger and independent sample set with a sensitive and quantitative method (i.e. ELISA) in multiple, well characterized individuals. Thus, this proof-of-principle study demonstrates that this proteomic approach can be used to reliably identify AD biomarkers in CSF.

The proteomic approach taken included multi-affinity depletion, 2-D DIGE, and nano-LC-FTMS. The sensitive, high resolution mass spectrometry resulted in the identification in 16 of the 18 gel features that showed significant differential abundances between the two CDR groups. The overall result was the generation of reliable data, which is demonstrated by the low false discovery rate (FDR) in the study. FDR is defined as the number of expected false predictions divided by the number of total predictions, which in the study is 21. The number of expected false predictions was estimated using a previous set of CSF proteomic data (generated using the same approach and performed in the same lab by the same people). A “same-same” comparison was approximated by comparing a set of 6 CSF samples from 8 individuals to a second set of 6 CSF samples from the same individuals, taken 2 weeks apart. Any differentially expressed proteins found in such a comparison would be false positives because in the ideal scenario, there should be no differences detected in a same-same comparison. Interestingly, no protein spots (0 out of 306 matched spots) were found that were significantly different between the two groups. With a close-to-zero FDR when the p value cutoff is set at 0.05, the p value cutoff could potentially be increased to maximize the number of biomarker candidates discovered while still maintaining a low FDR.

Four of the 6 selected biomarker candidates were validated using ELISA with a much larger characterized sample set. Although protein spots that correspond to GSN and AGT display differential abundance in 2D-DIGE, a difference between the mild AD and control group via ELISA was not detected. One possible explanation is the existence of different isoforms for a single protein. GSN has two isoforms “a” and “b” which differ only by a stretch of 24 amino acids at the N terminus. The unique peptides for the two isoforms were not defected in the MS data and the ELISA is not isoform specific. AGT is known to have different isoforms due to glycosylation Matsubara et al. Ann Neurol (1990) 28:561-67). If the 2D-DIGE observations only reflect differences in certain isoforms, it might not result in a difference in total protein level measured by ELISA. Thus, to the extent that AD is associated with changes in the level of specific protein isoforms, ELISA for total protein levels might not detect such changes.

One of the validated candidate biomarkers, ACT, has been previously proposed as a possible biomarker for AD. An increase in CSF ACT level was observed in the AD group, in agreement with a number of previous studies (Matsubara et al., Ann Neurol (1990) 28:561-67; DeKosky et al., Ann Neurol (2003) 53:81-90; Harigaya et al., Intern Ned (1995) 34:481-84; Licastro et al., Alzheimer Dis Assoc Disord (1995) 9:112-118). There was no significant change in ACT levels in plasma in the dementia group nor a correlation between the CSF and plasma levels of this protein. This is similar to some previous findings [33, 36-39] (Matsubara et al., Ann Neurol (1990) 28:561-67; Licastro et al., Alzheimer Dis Assoc Disord (1995) 9:112-118; Pirttila et al., Neurobiol Aging (1994) 15:313-317; Licastro et al., Dement Geriatr Cogn Disord (2000) 11:25-28; Lanzrein et al., Alzheimer Dis Assoc Disord (1998) 12:215-227) but not others (Matsubara et al., Ann Neurol (1990) 28:561-67; DeKosky et al., Ann Neurol (2003) 53:81-90; Licastro et al., Alzheimer Dis Assoc Disord (1995) 9:112-118; Oishi et al., Ann Clin Lab Sci (1996) 26:340-45; Licastro et al., J Nuroimmunol (2000) 103:97-102). The ACT index (a calculated quotient using the ACT and albumin content of CSF and plasma to detect intrathecal ACT synthesis) was also measured and it was found that 90% of the samples have an index above 1, with an average index of 2.4. These results indicate independent within-CNS production as the main source of ACT in CSF,

In contrast to ACT, other validated candidates (i.e. ATIII, ZAG and CNDP1) have not been well-studied in AD and therefore represent novel potential biomarkers. ATIII, like ACT, is a serine protease inhibitor. In the present and another study (Kalaria et al., Am J Pathol (1993) 143:886-893), ATIII was shown to localize to both amyloid plaques and NFT in AD brain. However, ATIII has not previously been reported to be increased in AD CSF. It is not clear why ZAG and CNDP1 are altered in AD CSF or their potential roles in AD pathogenesis. ZAG is a soluble glycoprotein and present in a variety of body fluids (Poortmans et al., J Lab Clin Med (1968) 71:807-11). The biological functions of ZAG are largely unknown. One 2D-gel-based proteomic study identified ZAG as one of the proteins that was increased in AD CSF (Davidsson et al., Neuroreport (2002) 13:611-15) though this was not validated in a large sample set. Carnosinase I (CNDP1) hydrolyzes carnosine (β-alanlyl-L-histidine) and homocamosine, (γ-3minobutyryl-L-histidlne), both of which are believed to be neuro-protective (Teufel et al., J Biol Chem (2003) 278:6521-31). Decreased serum levels of CNDP1 have been found in patients with Parkinson's disease, multiple sclerosis and in patients after a cerebrovascular accident (Wassif et al., Clin Chim Acta (1994) 225:57-64).

The degree of the changes of these validated biomarker candidates (in AD CSF) is moderate (within 30%), with overlaps between the AD and control group. However, the magnitude of the difference in levels of ACT, ATIII and ZAG is comparable to that of Aβ42, currently one of the best CSF biomarkers of AD that correlates very well with the presence or absence of amyloid in the brain (Fagan et al., Ann Neurol (2006) 59:512-19). Importantly, changes in these biomarkers are already evident in the very mildly demented CDR 0.5 group (FIG. 7), suggesting the suitability of these candidates for the early detection of AD. Due to the existence of preclinical AD in cognitively normal individuals, the CDR 0 group is likely heterogeneous, containing subjects with and without AD pathology in the brain, indeed, a recent study demonstrated the presence of brain amyloid and low levels of CSF Aβ42 in a subset of cognitively normal individuals (Fagan et al., Ann Neurol (2008) 59:512-19). Such preclinical AD pathology might in part account for the overlap between AD and control group in terms of biomarker concentrations.

Statistical analyses demonstrate that the biomarker candidates identified have diagnostic power/accuracy comparable to that of Aβ42 and total tau. When all biomarkers are combined, a greater AUC and sensitivity can be achieved. Although the difference is not statistically significant in this study (including 50 CDR 0 subjects and 33 mild AD subjects), this trend may become significant when larger sample sets are assessed.

1-88. (canceled)

89. A biomarker for Alzheimer's disease, wherein the biomarker is selected from the group consisting of the level of antithrombin III and the level of carnosinase in a bodily fluid of a subject.

90. The biomarker of claim 89, wherein the bodily fluid is CSF.

91. The biomarker of claim 89, wherein antithrombin III is selected from the group consisting of antithrombin III enzymatic activity and antithrombin III protein concentration, and carnosinase is selected from the group consisting of carnosinase enzymatic activity and carnosinase protein concentration.

92. A method for detecting AD in a subject, the method comprising:
a. quantifying the level of antithrombin III in a bodily fluid of the subject and determining if the quantified level of antithrombin III is elevated in comparison to the average antithrombin III level for a subject with a CDR of 0; or
b. quantifying the level of carnosinase in a bodily fluid of the subject, and determining if the quantified level of carnosinase is elevated in comparison to the average carnosinase level for a subject with a CDR of 0.

93. The method of claim 92, wherein an elevated antithrombin III level in comparison to the average antithrombin III level indicates a diagnosis of AD, or an elevated carnosinase level in comparison to the average carnosinase level indicates a diagnosis of AD.

94. The method of claim 92, wherein an elevated antithrombin III level in comparison to the average antithrombin III level indicates a prognosis of AD, or an elevated carnosinase level in comparison to the average carnosinase level indicates a prognosis of AD.

95. The method of claim 92, wherein the antithrombin III quantified is selected from the group consisting of antithrombin III enzymatic activity and antithrombin III protein concentration, and the carnosinase quantified is selected from the group consisting of carnosinase enzymatic activity and carnosinase protein concentration.

96. The method of claim 92, wherein the subject is selected from the group of subjects consisting of a subject at risk for AD and a subject that has no clinical signs of AD.

97. The method of claim 92, wherein the subject is human.

98. The method of claim 92, wherein the bodily fluid is CSF.

99. The method of claim 92, further comprising
a. quantifying the level of at least one other Alzheimer's disease biomarker in a bodily fluid of the subject, and
b. determining if the quantified level of the additional Alzheimer's disease biomarker or biomarkers is elevated or depressed in comparison to the average level for that biomarker for a subject with a CDR of 0.

100. The method of claim 99, further comprising selecting the additional biomarker or biomarkers from the group consisting of ACT, ZAG, ATIII, CNDP1, Aβ42, and tau.

101. A method for monitoring Alzheimer's disease, the method comprising:
a. quantifying the level of antithrombin III in a bodily fluid of the subject, and comparing the quantified level of antithrombin III to a previously quantified antithrombin III level of the subject, or
b. quantifying the level of carnosinase in a bodily fluid of the subject, and comparing the quantified level of carnosinase to a previously quantified carnosinase level of the subject.

102. The method of claim 101, where the subject's response to Alzheimer's disease treatment is monitored.

103. The method of claim 101, wherein the antithrombin III quantified is selected from the group consisting of antithrombin III enzymatic activity and antithrombin III protein concentration, and the carnosinase quantified is selected from the group consisting of carnosinase enzymatic activity and carnosinase protein concentration.

104. The method of claim 101, wherein the subject is selected from the group of subjects consisting of a subject diagnosed with AD and a subject that has no clinical signs of AD.

105. The method of claim 101, wherein the subject is human.

106. The method of claim 101, wherein the bodily fluid is CSF.

107. The method of claim 101, further comprising
a. quantifying the level of at least one other biomarker in a bodily fluid of the subject, and
b. comparing the quantified level of the additional AD biomarker or biomarkers to a previously quantified level of the subject for that biomarker.

108. The method of claim 107, further comprising selecting the additional biomarker or biomarkers from the group consisting of ACT, ZAG, ATIII, CNDP1, Aβ42, and tau.
